(19) United States
(12) Patent Application Publication
(10) Pub. No.: US 2026/0360508 A1
(43) Pub. Date: Nov. 12, 2026

(54) SYSTEM AND METHOD FOR MARKETPLACE-BASED FULFILLMENT OF PERSONALIZED MEAL PLANS

(71) Applicant: Peter Moses Bell, Ocala, FL (US)

(72) Inventor: Peter Moses Bell, Ocala, FL (US)

(21) Appl. No.: 19/675,472

(22) Filed: May 12, 2026

Related U.S. Application Data
(60) Provisional application No. 63/804,531, filed on May 12, 2025.

(51) Int. Cl. G16H 20/60; G06Q 30/08

(52) U.S. Cl. CPC G16H 20/60; G06Q 30/08

(57) ABSTRACT

A computer-implemented system and method for managing the fulfillment of personalized meal plans is disclosed herein. The system receives a personalized meal plan, such as a text file or image, and its meal plan processing engine generates a structured, machine-readable fulfillment request by parsing the plan to extract ingredients and dietary constraints. A provider matching engine filters a plurality of providers to identify an eligible set based on criteria such as location and dietary capabilities. A bidding and ranking engine transmits the request to the eligible providers, receives competitive bids, and ranks the bids based on multiple factors including price and provider rating. The ranked bids are presented to a client for selection, thereby enabling the end-to-end transformation of a personalized plan into a competitively fulfilled meal service. In some embodiments, the system includes modules for escrow-based payment and constraint-based ingredient substitution validation.

### STATEMENT REGARDING FEDERALLY SPONSORED RESEARCH OR DEVELOPMENT

This invention has been created without the sponsorship or funding of any federally sponsored research or development program.

### FIELD OF INVENTION

The present invention relates generally to computer-implemented marketplaces, and more specifically to systems and methods for transforming personalized meal plans into structured, fulfillable service requests for execution within a competitive provider marketplace.

### BACKGROUND

In the fields of nutrition, health, and fitness, personalized meal plans are a common tool used by dietitians, trainers, and health-conscious individuals to achieve specific dietary goals. These plans dictate what a person should eat, often including specific ingredients, quantities, and macronutrient targets. While the creation of such plans is well-established, a significant “execution gap” exists. Many individuals receive or create a detailed meal plan but lack the time, culinary skill, access to ingredients, or motivation to consistently prepare the specified meals.

Existing technological solutions fail to adequately bridge this gap. One category of solutions includes meal planning software. These applications can help a user generate a meal plan and a corresponding grocery list but offer no further support. The burden of shopping, preparing, and cooking the meals remains entirely on the user.

Another category includes meal kits and pre-prepared meal delivery services. These services provide convenience but lack true personalization. They typically operate from a fixed or rotating menu of pre-designed meals. A user must choose from what is offered, rather than having meals prepared according to their own externally-generated, personalized nutritional plan. These systems are not configured to ingest and execute a bespoke plan or a custom fit plan based on the individual user's needs and/or preferences.

A third category includes general service marketplaces or local cook marketplaces. While these platforms connect consumers with service providers, they lack the specialized, domain-specific functionality required for personalized nutrition. They are not equipped to parse, understand, and validate the complex dietary constraints, ingredient specifications, and nutritional data embedded in a personalized meal plan. They operate as generic request-and-quote platforms without the structured transformation, constraint-based validation, and controlled fulfillment logic needed to reliably and safely execute personalized meal plans.

Therefore, there is a need for a system that bridges the gap between personalized meal planning and meal fulfillment. A need exists for a technology platform that can transform a personalized meal plan into a structured, actionable service request and manage its fulfillment through a controlled and competitive marketplace of qualified providers.

### SUMMARY OF INVENTION

The present invention provides a computer-implemented system and method for managing the end-to-end fulfillment of personalized meal plans. The invention overcomes the limitations of the prior art by providing a platform that intelligently transforms a personalized meal plan into a structured fulfillment request and orchestrates its execution within a controlled, constraint-based provider marketplace.

According to one aspect, a computer-implemented system comprises a processor and memory configured with several engines. A meal plan processing engine receives a personalized meal plan and generates a structured, machine-readable fulfillment request by parsing the plan to extract ingredients, dietary constraints, and other parameters. A provider matching engine filters a plurality of providers to identify a set of eligible providers based on criteria relevant to the request, such as dietary capabilities and location. A bidding and ranking engine transmits the request to the eligible providers, receives competitive bids, and ranks the bids based on multiple factors such as price, provider rating, and distance. The ranked bids are then presented to a client for selection.

In certain embodiments, the system includes a substitution approval engine that allows providers to propose ingredient substitutions, automatically validates these substitutions against the client's dietary constraints, and routes them for approval. In further embodiments, the system includes an escrow fulfillment engine that secures client payment upon provider selection and releases the funds only upon confirmation that the order has been satisfactorily completed, thereby protecting both parties.

The present invention provides a technical solution to a technical problem by creating a specific, structured data object from unstructured or semi-structured input, and using that data object to control a multi-step workflow involving data filtering, competitive bid evaluation, and transaction management within a distributed network of users and providers.

### BRIEF DESCRIPTION OF THE DRAWINGS

FIG. 1 illustrates a high-level system architecture overview, according to an embodiment of the invention;

FIG. 2 illustrates the integration of a plurality of modules or engines that form the core infrastructure of the invention;

FIG. 3 is a flowchart illustrating a representative end-to-end workflow thereof;

FIG. 4 is a flowchart illustrating the order lifecycle states of the invention;

FIG. 5 is a flowchart illustrating the meal plan processing and transformation workflow thereof;

FIG. 6 is a flowchart illustrating the provider matching and filtering workflow feature of the invention;

FIG. 7 is a flowchart illustrating the bidding and ranking workflow feature of the invention;

FIG. 8 is a flowchart illustrating the payment and escrow workflow feature of the invention;

FIG. 9 is a flowchart illustrating the ingredient substitution workflow feature of the invention;

FIG. 10 is a flowchart illustrating delivery and pickup fulfillment options workflow of the invention;

FIG. 11 is a flowchart illustrating the dispute resolution workflow feature of the invention.

### DESCRIPTION OF THE INVENTION

Before the subject invention is described further, it is to be understood that the invention is not limited to the embodiments of the invention described below, as variations of the particular embodiments may be made and still fall within the scope of the invention. It is also to be understood that the terminology employed is for the purpose of describing particular embodiments and is not intended to be limiting.

The details of one or more embodiments of the invention are set forth in the accompanying drawings and the description below. The invention provides a computer-implemented platform that transforms personalized meal plans into structured, actionable fulfillment requests and manages their execution through a controlled, competitive marketplace.

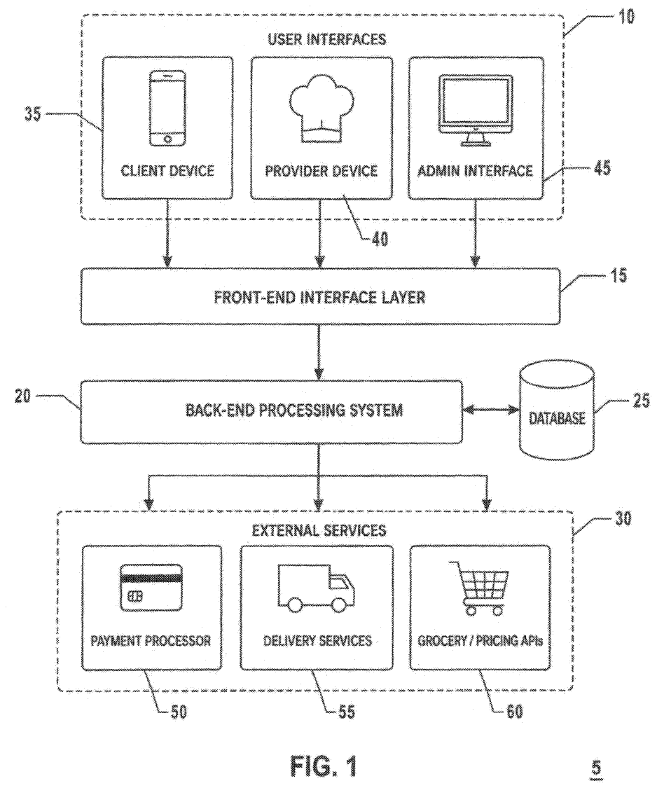

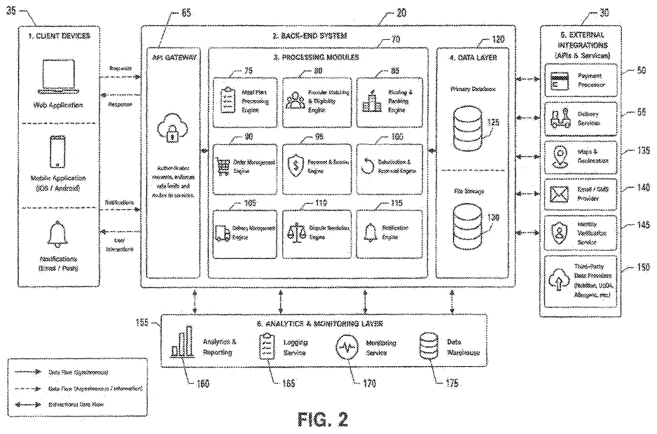

Referring to FIG. 1 and FIG. 2, the system architecture or platform 5 includes several key components. There are a plurality of systems, interfaces, and databases 10, 15, 20, 25, and 30. As shown in FIG. 1, users interact with the platform 5 through Client Devices 35, Provider Devices 40, or an Admin Interface 45 which then create a front-end interface layer 15. The front-end layer 15 and in turn the user interfaces 35, 40, and 45 may be web-based or mobile applications running on network-connected devices. These devices communicate with a Back-End Processing System 20 via the Front-End Interface Layer 15 and an API Gateway 65. The Back-End Processing System 20 is connected to a Database 25 for data storage and easy flow of information between the various interfaces and systems, including external services 30 for the processing of transactions/payments 50, delivery services or logistics 55, and pricing and inventory APIs 60.

The Back-End Processing System 20 comprises the core business logic 70 of the invention, implemented as a plurality of processing modules or engines executed by one or more processors. As shown in FIG. 2, these may include a Meal Plan Processing Engine 75, a Provider Matching and Eligibility Engine 80, a Bidding and Ranking Engine 85, an Order Management Engine 90, a Payment and Escrow Engine 95, and a Substitution and Approval Engine 100, a Delivery Management Engine 105, a Dispute Resolution Engine 110, and a Notification Engine 115 among others. This Back-End Processing System 20 interacts with a Data Layer 120, which may include a primary database 125 for transactional data and a file storage database 130 for documents and images. The system also integrates with External Services 30, such as payment processors 50, delivery services 55, maps and geolocation services 135, email and/or SMS providers 140, an Identity Verification Service 145, and third-party data providers 150 for accessing nutritional information (e.g. allergens, FDA statistics, USDA statistics etc.). The system also integrates with and an API Gateway 65 to manage authentication requests, enforces data transfer rate limits, and routes to services. The Back-End Processing System 20 directly interacts with the Analytics and Monitoring Layer 155 which in turn indirectly interacts with the flow of information across the Client Devices 35, the Data Layer 120, and the External Integrations 30. The Analytics and Monitoring Layer 155 includes functions such as Analytics and Reporting 160, a Logging Service 165, a Monitoring Service 170, and a Data Warehouse 175. The Analytics and Reporting 160 function generates operational reports, performance metrics, and business insights for delivering optimal and improved outputs. The Logging Service 165 captures system logs, audits, and security events. The Monitoring Service 170 monitors the overall health of the system, alerts, uptime, and performance. The Data Warehouse 175 aggregates data for trend analysis and machine learning in order to deliver optimal and improved outputs.

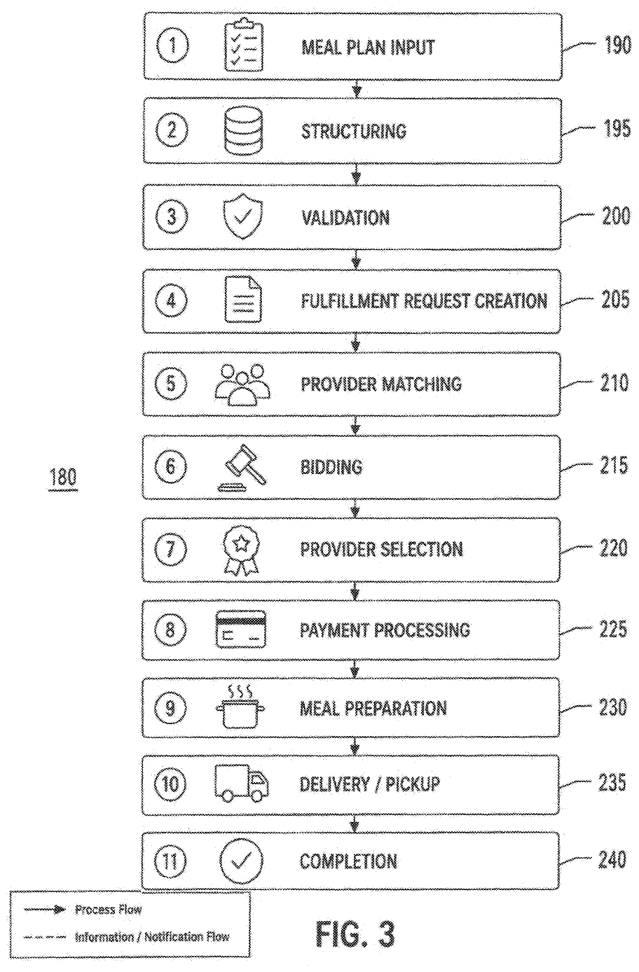

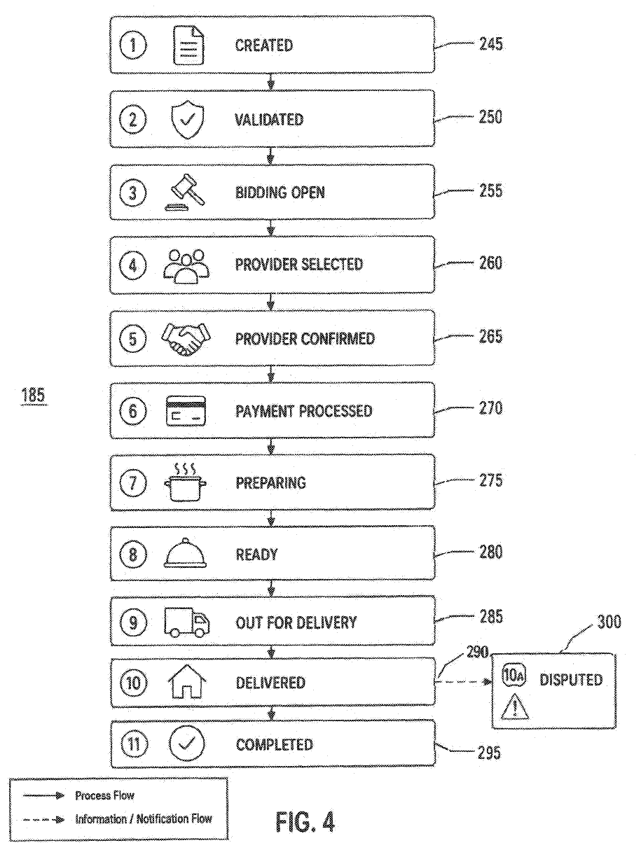

The overall workflow is illustrated in FIG. 3 and FIG. 4. FIG. 3 illustrates an overview of the End-to-End Workflow 180 and FIG. 4 illustrates an overview of the Order Lifecycle workflow 185. The End-to-End Workflow 180, as shown in FIG. 3, begins with a Meal Plan Input 190 whereby a user creates or uploads a personalized meal plan, then the workflow continues with Structuring 195 whereby the system parses and configures the Meal Plan Input 190 into machine-readable data. The Meal Plan Input 190 may be in various formats, such as typed text, a PDF document, or an image file. Then the next step in the workflow is Validation 200 whereby the system validates ingredients, macros, dietary rules, and dietary constraints before proceeding to Fulfillment Request Creation 205 at which point a structured request is created and published to the marketplace. Then the next step is Provider Matching 210 whereby the system identifies and notifies eligible providers of structured fulfillment requests, which in turn prompts a Bidding 215 process whereby multiple providers submit bids including price, timeframe, and other terms for a fulfillment request. Once all eligible bids have been submitted, the bids are ranked based on criteria such as pricing, location, ingredients; and then Provider Selection 220 is initiated whereby a user selects a preferred provider based on previously ranked bids. Once Provider Selection 220 is completed, then Payment Processing 225 occurs allowing a user to enter a method of payment and the payment is held in escrow. The completion of Payment Processing 225 prompts Meal Preparation 230 whereby the system alerts the selected provider to take steps to prepare meals according to the Meal Plan Input 190, including but not limited sourcing ingredients. Once the Meal Preparation 230 is completed, then the meals are scheduled for Delivery/Pickup 235 by a user. Upon the user receiving the personalized meals, the payment held in escrowed is released to the selected provider, marking Completion 240 of the End-to-End Workflow 180.

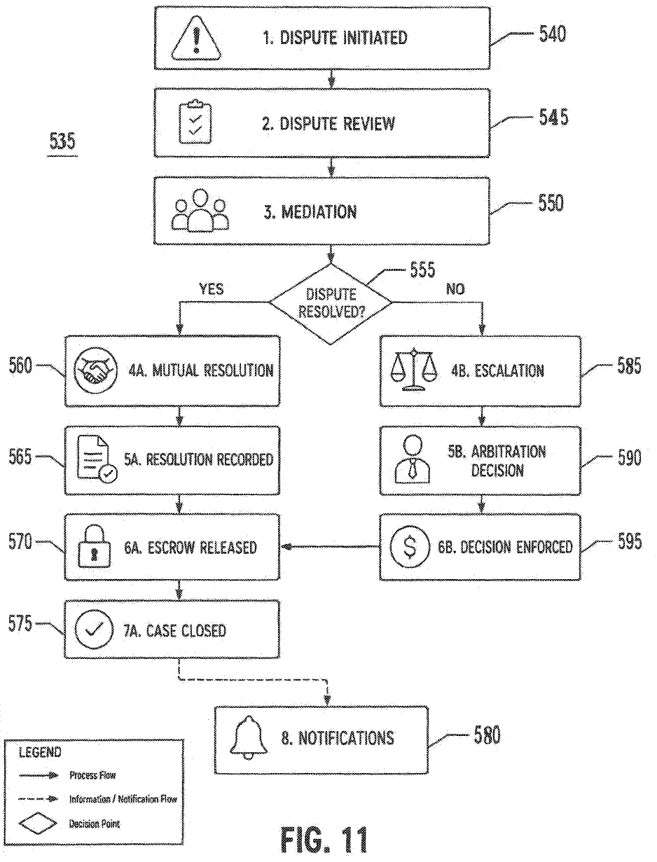

The system manages End-to-End Workflow 180 through a series of defined Order Lifecycle states 185, as shown in FIG. 4. A Meal Plan Input 190 proceeds through the Order Lifecycle 185 starting from Created 245, to Validated 250, to Bidding Open 255, Provider Selected 260, to Provider Confirmed 265, to Payment Processed 270, to Preparing 275, to Ready 280, to Out for Delivery 285, to Delivered 290, and finally to Completed 295. At the Delivered 290 Order Lifecycle state 185, there is a built in Dispute state 300 whereby a user has the option to report an issue with a meal that was delivered/received, opening a dispute to resolve the issue before moving to the Completed 295 state. The Dispute state 295 is described and illustrated in greater detail below and in FIG. 11. The Order Lifecycle states 185 provides granular tracking and control over the meal plan fulfillment process or workflow.

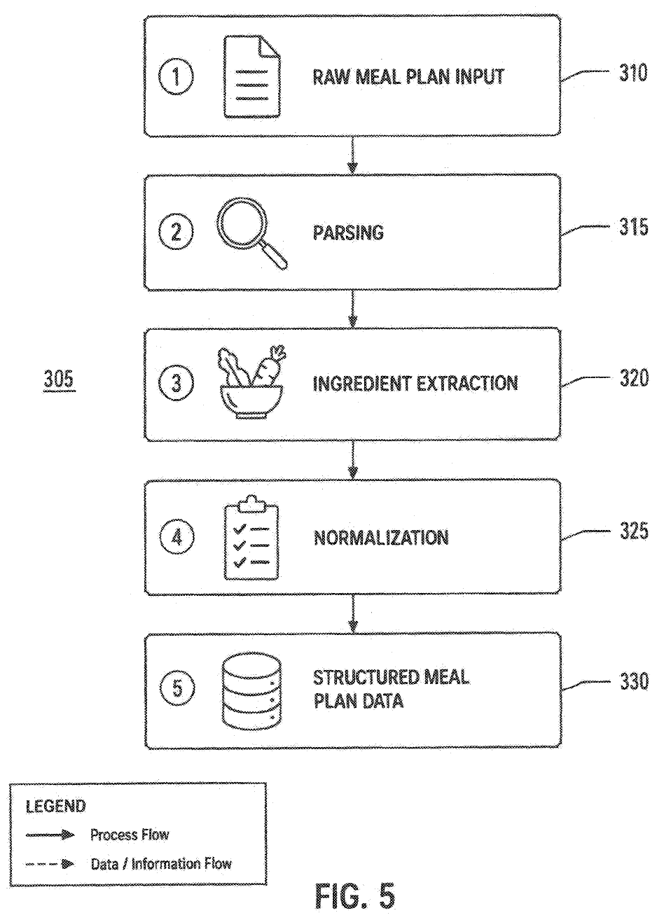

As shown in FIG. 5, a Meal Plan Processing Engine 305 works in the background to achieve steps to formulate and customize the Meal Plan Input 190 of the End-to-End Workflow 180. The Meal Plan Processing Engine 305 receives the Raw Meal Plan Input 310 and implements Parsing 315 to analyze the input and identify meals, ingredients, quantities, and other elements for the Meal Plan Input 190. The next step is Ingredient Extraction 320 whereby the ingredients and associated attributes are extracted from the Parsed 315 content of the Meal Plan Input 190. This is followed by a Normalization 325 step which standardizes the extracted data, for example by converting units and mapping ingredient names to a categorization system. The Meal Plan Processing Engine 305 final output is Structured Meal Plan Data 330 in the form of a machine-readable data object that serves as the foundation for Fulfillment Request Creation 205 and corresponds with the Structuring 195 step of the End-to-End Workflow. This normalized data is stored as structured records for Validation 200, Matching 210, and Fulfillment Request Creation 205. The work carried out by the Meal Plan Processing Engine 305 enables the meal plan to function as an actionable service request or Fulfillment Request Creation 205 rather than a static document.

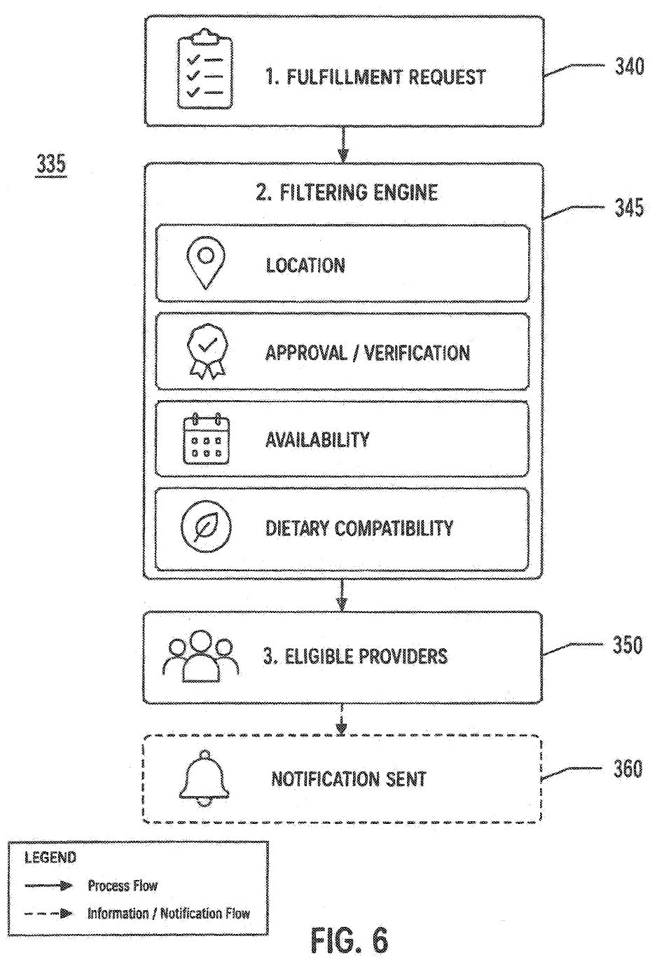

As shown in FIG. 6 a Provider Matching Engine 335 works in the background to accomplish the Provider Matching 210 process of the End-to-End Workflow 180 (FIG. 3). The process begins with completing Validation 200 and the Fulfillment Request Creation 205 (FIG. 3), then continues with generating a Fulfillment Request 340, which is generated from the Structured Meal Plan Data 330. Once the Fulfillment Request 340 has been created, a Filtering engine 345 applies multiple filters to identify eligible providers. The Filter Engine 350 analyzes and sets certain parameters for eligibility including location, approval/verification, availability, and dietary compatibility. This list of parameters are exemplary and not intended to be exhaustive or limiting. A provider must be within an acceptable delivery, pick up, and/or service area range of a user submitting the Meal Plan Input 190. The service area range may optimally be within a 5 to 10 mile radius, however the shorter or longer distances may be available depending on the Meal Plan Input 190 and the resulting Fulfillment Request 345. Providers must be approved and verified to meet platform or system requirements, and also must be available during the requested time window. Examples of system requirements may include but is not limited to local, state, or federal government regulations, food handling permits, health guidelines, licenses to operate, compliance with training or certifications, etc. The time window requirements may include but is not limited to 2 to 3 days intervals for fulfillment by providers. Dietary compatibility (e.g., ability to handle gluten-free or vegan requests) is of utmost importance for determining provider eligibility and therefore providers must be able to fulfill a user's dietary rules, ingredients constraints, and health goals. Once providers pass through and comply with the multiple filters of the Filtering engine 345, then Eligible providers 350 are returned by the system and are added to the Bidding 215 process. The Provider Matching Engine 335 sends Notification 355 to the filtered Eligible providers 350 that a new meal plan request is available for Bidding 215.

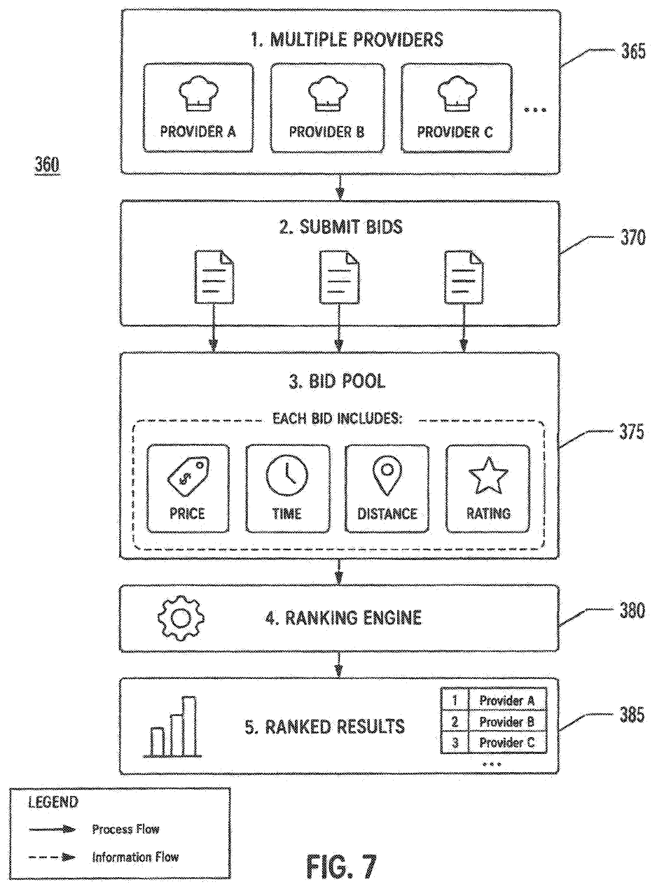

Next, as shown in FIG. 7, a Bidding and Ranking Engine 360 manages a competitive Bidding 215 process. Multiple Eligible Providers 365 are invited to Submit Bids 370 with proposed terms and conditions and then become part of a Bid Pool 375. Each bid in the Bid Pool 375 may include but is not limited to various terms, such as the proposed price (cost for fulfillment), estimated preparation/delivery time, distance from provider to user/client, and the ability for a user to rate and review providers. Once all received bids with terms and conditions are collected in the Bid Pool 375, then a Ranking Engine 380 evaluates the bids based on a plurality of these factors to generate Ranked Results 385, which are presented to the client for selection. This multi-factor ranking provides a more sophisticated decision-making tool than simple price comparison. The Ranked Results 385 provide a user with important and useful information that closely aligns with their Meal Plan Input 190.

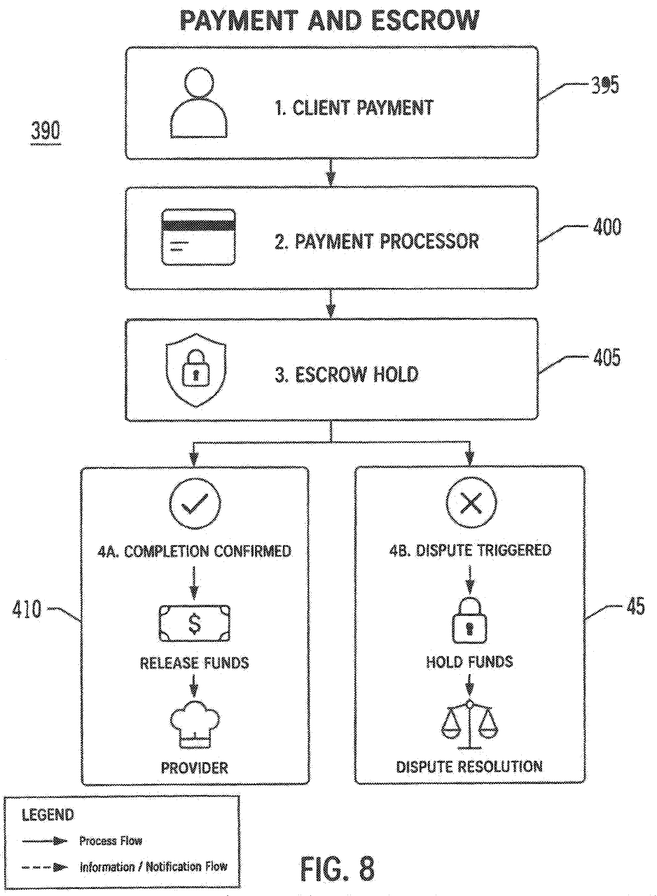

As shown in FIG. 8, a Payment and Escrow Engine 390 manages and processes Client Payment 395 once the client reviews an order and authorizes payment for an Eligible Provider 350 from the Ranked Results 385 generated by the Ranking and Bidding Engine 360. A Payment Processer 400 securely processes the authorized payment and transfers funds into an escrow account, creating an Escrow Hold 405. If the client is satisfied with the provider and completion of the order, then a Completion Confirmed 410 input from the client prompts the release of the Escrow Hold 405 and the provider receives the agreed upon Client Payment 395. However, if the client is not satisfied with the provider and completion of the order, then a Dispute 415 is triggered where the client has the ability and opportunity to report an issue with the order and/or the provider. The Dispute 415 input from the client keeps the Escrow Hold 405 in place, and the funds remain in escrow while the Dispute 415 is reviewed and resolved. Once the Dispute is resolved, resulting in a Completion Confirmed 410 input from the client, release of the Escrow Hold 405, and the provider receives the agreed upon Client Payment 395. In the event that the Dispute 415 is not resolved, the funds are released back to the client for a full refund or applied towards credit for a future order.

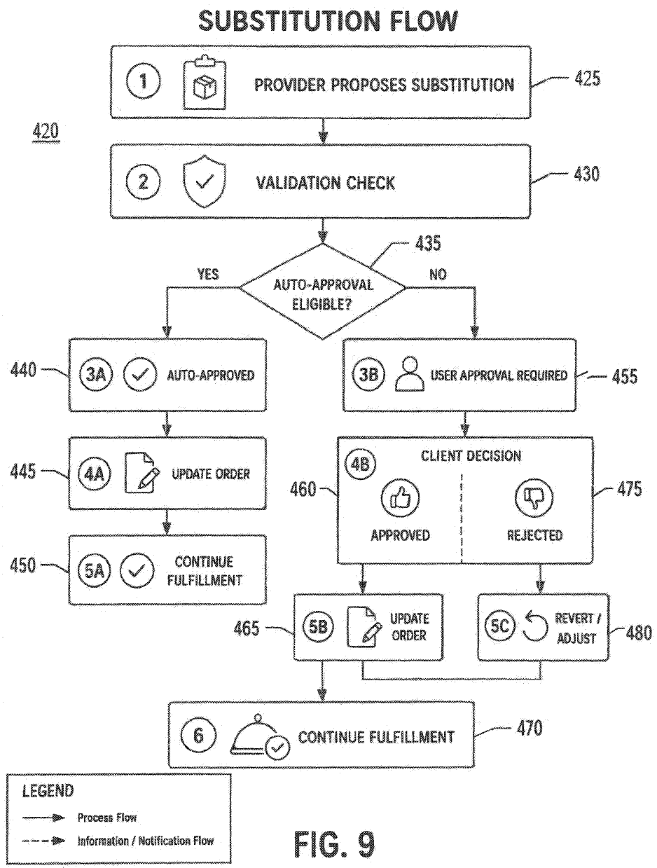

In some embodiments of the invention, the system facilitates a provider substitution workflow as shown in FIG. 9. A Substitution Flow Engine 420 manages the instance when a provider may propose a substitution 425 for an unavailable ingredient. The Substitution Flow Engine 430 performs a Validation Check 430 against the Structured Meal Plan Data 330 to ensure the substitute does not violate the client's dietary constraints, allergies, or nutritional targets. If the Validation Check 430 returns a compliant substitution, then the Substitution Flow Engine 420 must determine whether the substitution is Auto-Approval Eligible 435. If the substitution is eligible for auto-approval 435 based on predefined rules, it is Auto-Approved 440. The Auto-Approved 440 substitution moves to the next step to Update Order 445 and then Continue Fulfillment 450. Otherwise, if the substitution is not Auto-Approval Eligible 435, it is routed to the client for a decision. A User Approval Required 455 prompt is generated, and then if the substitution is Approved 460 by the client, the process moves to Update the Order 465 with the substitution, and then Continue to Fulfillment 470 based on the final decision and updated order 465. However, if upon receiving the User Approval Required 455 prompt, the substitution is Rejected 475 by the client, then the system Reverts/Adjusts 480 to the original item or the provider selects an alternative, and the order is updated accordingly to Continue Fulfillment 470.

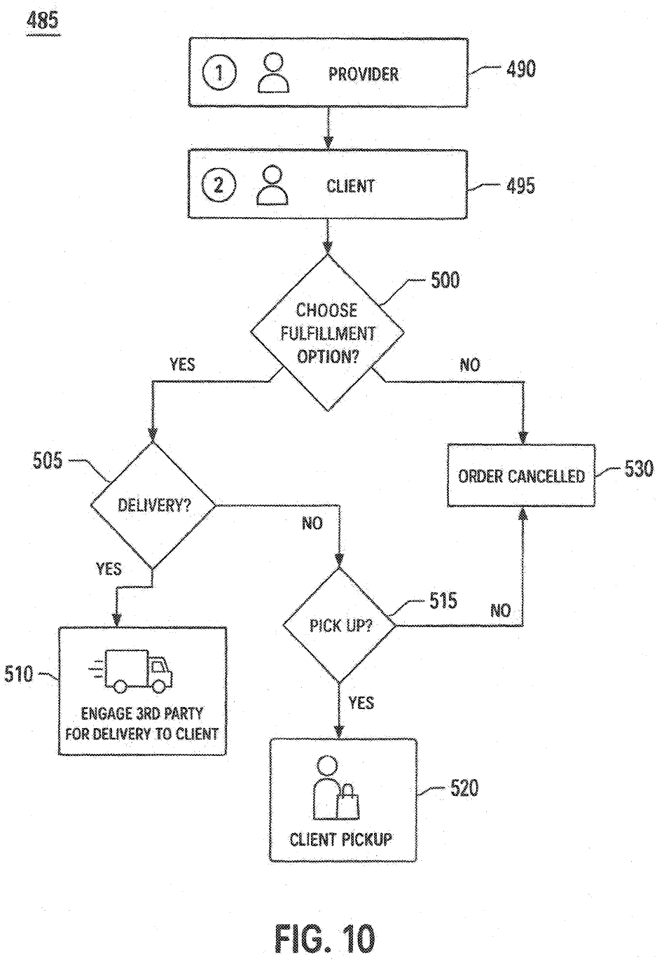

As shown in FIG. 10, a Delivery Flow Engine 485 manages the delivery/pickup 235 process (FIG. 3). The process begins when a Provider 490 prepares a meal or multiple meals and then confirms with a client 495 that the meal(s) is/are ready for fulfillment. The client 495 is alerted that the order is ready, and is prompted to choose a fulfillment option 500 for retrieving the prepared meal(s). If the client decides to choose a fulfillment option, then he is prompted to select a delivery option 505, and if he selects the delivery option 505, then the system engages a third party delivery service 510 to deliver the meal(s) to the client at a confirmed location. If the client does not choose the delivery option 505, then he is prompted to choose a pickup option 515, and if the client chooses the pickup option 515, then he will pick up 520 the meal(s) at an agreed upon location. Once the client 495 receives the order and confirms delivery in the system 410 (e.g. mobile or web-based), the Escrow Hold 405 is released and the process reaches Completion 240. If the client does not choose a fulfillment option 500 or does not choose the Pickup option 515, then the order is cancelled 530. If the order is cancelled 530, then the system will adjust or revert to processing a refund, issuing a credit, or the client may initiate a Dispute 300 which is processed by the Dispute Resolution Engine 535 (FIG. 11).

FIG. 11 illustrates the Dispute Resolution Engine 535 that manages the Dispute 300 process when a Dispute is Triggered 415 (See FIG. 4 and FIG. 8 respectively). As shown in FIG. 11, a Dispute is Initiated 540 when a client reports an issue with an order and opens a dispute in the system (e.g. mobile or web-based application). A Dispute Review 545 is initiated whereby the system notifies the provider of the issue, then both the client and the provider submit evidence and/or explanations in support of their respective positions on the issue. The dispute then moves to a Mediation 550 phase whereby a MacroExchange reviews and evaluates all available evidence and explanations, and is able to mediate between the client and the provider in order to resolve the issue. A MacroExchange for purposes of this invention refers to members of a corporate body or a panel of officers from a corporate entity responsible for governance and administration of infrastructure of this invention. Based on the results of inputted by the MacroExchange, the system determines whether the dispute is resolved 555. If there is a mutual resolution 560 wherein both parties reach a mutual agreement with the outcome of the dispute, then the resolution is recorded 565 in the system, the Escrow Hold is released 570, the dispute is closed 575 and the parties are individually notified 580 of final resolution. However, if there is no resolution by mutual agreement 560, the process moves to an Escalation 585 phase, and the parties must submit to arbitration. An arbitrator is assigned to the dispute, who then reviews all of the evidence and explanations provided during the Dispute Review 545 and Mediation 550 phase, and then makes a final decision 590 which is binding on both parties. The Arbitration Decision 590 is enforced 595 on both parties accordingly, and the Escrow Hold is released 570 and distributed based on the arbitration decision 590. The dispute is closed 575 and the parties are individually notified 580 of final resolution.

This invention is a computer-implemented system for managing the fulfillment of personalized meal plans, the system comprising: a processor; and a memory storing instructions that, when executed by the processor, configure the system to comprise: a meal plan processing engine configured to: receive a personalized meal plan for a client, the personalized meal plan comprising at least one meal with a corresponding set of ingredients and one or more dietary constraints; and generate a structured fulfillment request by parsing the personalized meal plan to create a machine-readable data object comprising the ingredients and the one or more dietary constraints; a provider matching engine configured to: filter a plurality of providers based on a set of eligibility criteria to identify a set of eligible providers, wherein the eligibility criteria are associated with the structured fulfillment request; and a bidding and ranking engine configured to: transmit the structured fulfillment request to the set of eligible providers; receive, from two or more of the eligible providers, a corresponding plurality of bids for fulfilling the structured fulfillment request; rank the plurality of bids based on a plurality of ranking factors to generate a set of ranked bids; and transmit the set of ranked bids to a client device associated with the client for selection of a provider.

The system of the invention as disclosed above wherein the meal plan processing engine is further configured to generate the structured fulfillment request by normalizing ingredient data extracted from the personalized meal plan and mapping the normalized ingredient data to a system taxonomy. The system of the invention as disclosed above, wherein the bidding and ranking engine is further configured to retrieve external ingredient pricing data from one or more third-party data sources and provide the external ingredient pricing data to the set of eligible providers to inform the generation of the plurality of bids. The system of the invention as disclosed above, wherein the set of eligibility criteria comprises at least one of: a geographic service radius, a provider approval status, provider availability, or a dietary preparation capability. The system of the invention as disclosed above, wherein the plurality of ranking factors comprises at least two of: a bid price, an estimated delivery time, a distance from the provider to the client, or a provider rating. The system of the invention as disclosed above, further comprising a substitution approval engine configured to: receive, from a selected provider, a proposal to substitute an ingredient in the structured fulfillment request; automatically validate the proposed substitution against the one or more dietary constraints contained within the machine-readable data object; and based on the validation, selectively route the proposal to the client device for approval. The system of the invention as disclosed above, further comprising an escrow fulfillment engine configured to: upon selection of a provider from the set of ranked bids, process a payment from the client and hold the payment in an escrow account; and upon receiving a confirmation of order completion, trigger a release of the payment from the escrow account to the selected provider.

This invention is a computer-implemented method for managing the fulfillment of personalized meal plans, the method comprising: receiving, by a computer system, a personalized meal plan for a client, the personalized meal plan comprising at least one meal with a corresponding set of ingredients and one or more dietary constraints; generating, by the computer system, a structured fulfillment request by parsing the personalized meal plan to create a machine-readable data object comprising the ingredients and the one or more dietary constraints; filtering, by the computer system, a plurality of providers based on a set of eligibility criteria to identify a set of eligible providers, wherein the eligibility criteria are associated with the structured fulfillment request; transmitting, by the computer system, the structured fulfillment request to the set of eligible providers; receiving, by the computer system, from two or more of the eligible providers, a corresponding plurality of bids for fulfilling the structured fulfillment request; ranking, by the computer system, the plurality of bids based on a plurality of ranking factors to generate a set of ranked bids; and transmitting, by the computer system, the set of ranked bids to a client device associated with the client for selection of a provider.

The method of the invention as disclosed above, wherein generating the structured fulfillment request further comprises normalizing ingredient data extracted from the personalized meal plan and mapping the normalized ingredient data to a system taxonomy. The method of the invention as disclosed above, further comprising retrieving external ingredient pricing data from one or more third-party data sources and providing the external ingredient pricing data to the set of eligible providers to inform the generation of the plurality of bids. The method of the invention as disclosed above, wherein the set of eligibility criteria comprises at least one of: a geographic service radius, a provider approval status, provider availability, or a dietary preparation capability. The method of the invention as disclosed above, wherein the plurality of ranking factors comprises at least two of: a bid price, an estimated delivery time, a distance from the provider to the client, or a provider rating. The method of the invention as disclosed above, further comprising: receiving a proposal from a selected provider to substitute an ingredient in the structured fulfillment request; automatically validating the proposed substitution against the one or more dietary constraints contained within the machine-readable data object; and based on the validation, selectively transmitting the proposal to the client device for approval. The method of the invention as disclosed above, further comprising managing an order lifecycle by transitioning a status of the structured fulfillment request through a plurality of defined states, the states including at least: a bidding state, a provider selection state, a meal preparation state, and a completed state. The method of the invention as disclosed above, further comprising: upon selection of a provider from the set of ranked bids, processing a payment from the client and holding the payment in an escrow account; and upon receiving a confirmation of order completion, triggering a release of the payment from the escrow account to the selected provider.

In alternative embodiments, the system may be configured with different models. For example, provider selection may occur through direct selection or automatic assignment rather than competitive bidding. Pricing may be fixed or subscription-based. Fulfillment may be handled by multiple providers who each prepare a portion of a single meal plan. These variations allow the core inventive concept to be adapted to different market conditions and business models while retaining the essential transformation and management logic.

As various changes may be made in the above-described subject matter without departing from the scope and the spirit of the invention, it is intended that all subject matter contained in the above description, or shown in the accompanying drawings, will be interpreted as descriptive and illustrative, and not in a limiting sense.

What is claimed is:

1. A computer-implemented system for managing the fulfillment of personalized meal plans, the system comprising: a processor; and a memory storing instructions that, when executed by the processor, configure the system to comprise: a meal plan processing engine configured to: receive a personalized meal plan for a client, the personalized meal plan comprising at least one meal with a corresponding set of ingredients and one or more dietary constraints; and generate a structured fulfillment request by parsing the personalized meal plan to create a machine-readable data object comprising the ingredients and the one or more dietary constraints; a provider matching engine configured to: filter a plurality of providers based on a set of eligibility criteria to identify a set of eligible providers, wherein the eligibility criteria are associated with the structured fulfillment request; and a bidding and ranking engine configured to: transmit the structured fulfillment request to the set of eligible providers; receive, from two or more of the eligible providers, a corresponding plurality of bids for fulfilling the structured fulfillment request; rank the plurality of bids based on a plurality of ranking factors to generate a set of ranked bids; and transmit the set of ranked bids to a client device associated with the client for selection of a provider.

2. The system of claim 1, wherein the meal plan processing engine is further configured to generate the structured fulfillment request by normalizing ingredient data extracted from the personalized meal plan and mapping the normalized ingredient data to a system taxonomy.

3. The system of claim 1, wherein the bidding and ranking engine is further configured to retrieve external ingredient pricing data from one or more third-party data sources and provide the external ingredient pricing data to the set of eligible providers to inform the generation of the plurality of bids.

4. The system of claim 1, wherein the set of eligibility criteria comprises at least one of: a geographic service radius, a provider approval status, provider availability, or a dietary preparation capability.

5. The system of claim 1, wherein the plurality of ranking factors comprises at least two of: a bid price, an estimated delivery time, a distance from the provider to the client, or a provider rating.

6. The system of claim 1, further comprising a substitution approval engine configured to: receive, from a selected provider, a proposal to substitute an ingredient in the structured fulfillment request; automatically validate the proposed substitution against the one or more dietary constraints contained within the machine-readable data object; and based on the validation, selectively route the proposal to the client device for approval.

7. The system of claim 1, further comprising an escrow fulfillment engine configured to: upon selection of a provider from the set of ranked bids, process a payment from the client and hold the payment in an escrow account; and upon receiving a confirmation of order completion, trigger a release of the payment from the escrow account to the selected provider.

8. A computer-implemented method for managing the fulfillment of personalized meal plans, the method comprising: receiving, by a computer system, a personalized meal plan for a client, the personalized meal plan comprising at least one meal with a corresponding set of ingredients and one or more dietary constraints; generating, by the computer system, a structured fulfillment request by parsing the personalized meal plan to create a machine-readable data object comprising the ingredients and the one or more dietary constraints; filtering, by the computer system, a plurality of providers based on a set of eligibility criteria to identify a set of eligible providers, wherein the eligibility criteria are associated with the structured fulfillment request; transmitting, by the computer system, the structured fulfillment request to the set of eligible providers; receiving, by the computer system, from two or more of the eligible providers, a corresponding plurality of bids for fulfilling the structured fulfillment request; ranking, by the computer system, the plurality of bids based on a plurality of ranking factors to generate a set of ranked bids; and transmitting, by the computer system, the set of ranked bids to a client device associated with the client for selection of a provider.

9. The method of claim 8, wherein generating the structured fulfillment request further comprises normalizing ingredient data extracted from the personalized meal plan and mapping the normalized ingredient data to a system taxonomy.

10. The method of claim 8, further comprising retrieving external ingredient pricing data from one or more third-party data sources and providing the external ingredient pricing data to the set of eligible providers to inform the generation of the plurality of bids.

11. The method of claim 8, wherein the set of eligibility criteria comprises at least one of: a geographic service radius, a provider approval status, provider availability, or a dietary preparation capability.

12. The method of claim 8, wherein the plurality of ranking factors comprises at least two of: a bid price, an estimated delivery time, a distance from the provider to the client, or a provider rating.

13. The method of claim 8, further comprising: receiving a proposal from a selected provider to substitute an ingredient in the structured fulfillment request; automatically validating the proposed substitution against the one or more dietary constraints contained within the machine-readable data object; and based on the validation, selectively transmitting the proposal to the client device for approval.

14. The method of claim 8, further comprising managing an order lifecycle by transitioning a status of the structured fulfillment request through a plurality of defined states, the states including at least: a bidding state, a provider selection state, a meal preparation state, and a completed state.

15. The method of claim 8, further comprising: upon selection of a provider from the set of ranked bids, processing a payment from the client and holding the payment in an escrow account; and upon receiving a confirmation of order completion, triggering a release of the payment from the escrow account to the selected provider.
